(19) United States
(12) Patent Application Publication
(10) Pub. No.: US 2012/0196928 A1
(43) Pub. Date: Aug. 2, 2012

(54) RED YEAST RICE EXTRACT HIGH IN MONACOLIN K CONTENT

(76) Inventor: Joar A. Opheim, Aptos, CA (US)

(21) Appl. No.: 13/443,736

(22) Filed: Apr. 10, 2012

Related U.S. Application Data
(63) Continuation of application No. 13/420,406, filed on Mar. 14, 2012.
Application No. 13/420,406 is a continuation-in-part of application No. 12/630,686, filed on Dec. 3, 2009.
Application No. 12/630,686 is a continuation-in-part of application No. 11/757,340, filed on Jun. 1, 2007.

(51) Int. Cl. A61K 31/366; A61P 3/06

(52) U.S. Cl. 514/460

(57) ABSTRACT

Compositions comprising Red Yeast Rice extracts which comprise greater than 5 wt. % monacolin K are useful for lowering cholesterol and/or triglyceride levels in a subject.

### 1. CROSS-REFERENCES TO RELATED APPLICATIONS

This application is a continuation of U.S. patent application Ser. No. 13/420,406, filed Mar. 14, 2012, which is a continuation-in-part of U.S. patent application Ser. No. 12/630,686, filed Dec. 3, 2009, which is a continuation-in-part of U.S. patent application Ser. No. 11/757,340, filed Jun. 1, 2007, both applications being incorporated herein by this reference in their entirety. All patents and patent applications cited in this application, all related applications referenced herein, and all references cited therein are incorporated herein by reference in their entirety as if restated here in full and as if each individual patent and patent application was specifically and individually indicated to be incorporated by reference.

### 2. BACKGROUND OF THE INVENTION

The invention relates to compositions and the use of such compositions in therapeutic compositions, nutritional supplements and medicaments, wherein the compositions are Red Yeast Rice extract, having hydroxymethylglutaryl-CoA (HMG-CoA) reductase inhibitor activity, wherein the Red Yeast Rice extract is high in monacolin K content.

Statins (which are members of a group of HMG-CoA reductase inhibitors) are a group of hypolipidemic agents, used as pharmaceutical agents to lower cholesterol levels in people with or at risk for cardiovascular disease. Statins lower cholesterol by inhibiting the enzyme HMG-CoA reductase, which is the rate-limiting enzyme of the mevalonate pathway of cholesterol synthesis. Inhibition of this enzyme in the liver stimulates low-density lipoprotein (LDL) receptors, resulting in an increased clearance of LDL, so-called “bad cholesterol,” from the bloodstream and a decrease in blood cholesterol levels.

Statins are potent cholesterol-lowering agents, and have been reported to lower LDL cholesterol by 30-50% (see Jones et al., “Comparative dose efficacy study of atorvastatin versus simvastatin, pravastatin, lovastatin, and fluvastatin in patients with hypercholesterolemia (the CURVES study),” Am J Cardiol 1998, 81-(5); 582-7.) Statins are classified as either synthetic or fermentation derived. Lovastatin was isolated from a strain of Aspergillus terreus and it was the first statin approved by the FDA as a drug (August 1987). Lovastatin is a water insoluble, white crystalline solid. The aqueous insolubility of lovastatin leads to inadequate dissolution in gastrointestinal fluids and, hence, poor absorption, distribution, and targeted organ delivery. Solubility of lovastatin is enhanced by reaction with 3-cyclodextrin, an oligosaccharide which improves the solubility of lovastatin. The improvement of aqueous solubility in such a case is a valuable goal to improve therapeutic efficacy. Lovastatin can also produce slight to moderate increases in high density lipoproteins (HDL) (10-20%), and slight decreases in triglycerides (5-10%). The usual daily dose of lovastatin is 20-80 mg/day. The statin drugs include lovastatin, pravastatin, fluvastatin, atorvastatin, simvastatin, rosuvastatin, and cerivastatin.

Compounds similar to lovastatin have also been found in a natural fermentation product known as Red Yeast Rice. These compounds are also HMG-CoA reductase inhibitors. A monograph published in Alternative Medicine Review (Volume 9, Number 2, 2004) reports that the HMG-CoA reductase inhibitor activity in Red Yeast Rice comes from a naturally occurring family of nine compounds called “monacolins,” each of which has HMG-CoA reductase inhibitor activity. Additional active ingredients in Red Yeast Rice include sterols (beta-sitosterol, campesterol, sigmasterol, and sapogenin), isoflavones, and monounsaturated fatty acids (see Heber et al., “Cholesterol lowering effects of proprietary Chinese red yeast rice dietary supplement,” Am J Clin Nutr 1999, 69:231-236. One of the monacolins in Red Yeast Rice, monacolin K, is said to be the lactone form of the statin drug lovastatin, which is converted to the active acid form in vivo by the liver.

Red Yeast Rice is a common foodstuff in Asian countries where the average daily intake is 14-55 grams. The nutritional supplement derived from Red Yeast Rice is known as Red Yeast Rice extract. It is obtained by drying the fermented product of rice on which the yeast Monascus pupureus has been grown and extracting the dried product with a solvent, usually aqueous ethanol or water. The Red Yeast Rice extract thus produced typically contains about 0.2 wt. % monacolin K and about 0.5 wt. % total monacolins.

U.S. Pat. No. 6,046,022, issued Apr. 4, 2000, to Zhang et al., discloses some methods of making high lovastatin Red Yeast Rice and using Red Yeast Rice and Red Yeast Rice extract. U.S. Pat. No. 6,046,022 is hereby incorporated by reference herein in its entirety.

U.S. Pat. No. 6,541,005, issued Apr. 1, 2003, to Yegorova; U.S. Pat. No. 6,436,406, issued Aug. 20, 2002, to Yegorova; U.S. Pat. No. 6,495,173, issued Dec. 17, 2002, to Yegorova; U.S. Pat. No. 6,544,525, issued Apr. 8, 2003, to Yegorova; U.S. Pat. No. 6,576,242, issued Jun. 10, 2003, to Yegorova; U.S. Pat. No. 6,541,006, issued Apr. 1, 2003, to Yegorova; and U.S. Pat. No. 6,410,521, issued Jun. 25, 2002, to Mundy et al., disclose methods of using Red Yeast Rice. All of these patents are incorporated herein by reference in their entirety.

U.S. Patent Application Publication No. 2006/0211763, published Sep. 21, 2006, by Fawzy et al., discloses a statin drug dissolved in a solvent system comprising natural or synthetic omega-3 fatty acids and U.S. Patent Application Publication No. 2006/0034815, published Feb. 16, 2006, to Guzman et al., discloses omega-3 oil solutions of one or more statins.

Fraser, 2001, Effect of Fish Oil and Red Yeast Rice Supplementation of Cardiovascular Disease Risk Factors in Hypercholesterolemic Men (Master's Thesis), retrieved from Dissertations and Theses Database (Publication ID MQ65930) discloses treatment of hypercholesterolemic men using fish oil and Red Yeast Rice, administered separately in separate capsules.

### 3. BRIEF SUMMARY OF THE INVENTION

In accordance with the present invention there is provided a composition comprising Red Yeast Rice extract comprising greater than 5 wt. % monacolin K. Also provided is such a composition wherein the Red Yeast Rice extract comprises 5.5 wt. % to about 25 wt. % monacolin K.

Further provided in accordance with the present invention is a composition wherein a daily dose of the composition comprises a sufficient amount of Red Yeast Rice extract to provide at least about 6.0 mg of monacolin K, about 6.4 mg of monacolin K, about 9.6 mg of monacolin K, about 12.8 mg monacolin K, about 14.4 mg monacolin K or about 16.0 mg monacolin K.

The present invention further provides a method of reducing serum cholesterol, triglycerides or both in a subject comprising administering to the subject an effective amount of a dosage comprising Red Yeast Rice extract comprising greater than 5 wt. % monacolin K. Also provided is such a method wherein the Red Yeast Rice extract comprises 5.5 wt. % to about 25 wt. % monacolin K.

Further provided in accordance with the present invention is such a method wherein a daily dose of the composition comprises a sufficient amount of Red Yeast Rice extract comprising greater than 5 wt. % monacolin K to provide at least about 6.0 mg of monacolin K, about 6.4 mg monacolin K, or about 9.6 mg monacolin K, about 12.8 mg monacolin K, about 14.4 mg monacolin K or about 16.0 mg monacolin K.

As used herein, the term “about” means that the value or amount to which it refers can vary by ±5%.

### 4. DETAILED DESCRIPTION OF THE INVENTION

The present invention relates to compositions or medicaments comprising Red Yeast Rice extract comprising greater than 5 wt. % monacolin K. In some embodiments, the compositions include those in which the Red Yeast Rice extract comprises 5.5 wt. % to about 25 wt. % monacolin K. These weight percentages are to be understood to include any weight percent or range of weight percentages greater than 5 wt %, including those between 5.5 wt % and 25 wt %, in increments of 0.01 wt %.

In some embodiments, the daily dose of the composition can comprise a sufficient amount of Red Yeast Rice extract comprising greater than 5 wt. % monacolin K to provide at least about 6.0 mg of monacolin K, or the daily dose can comprise a sufficient amount of such Red Yeast Rice extract, or the daily dose can comprise a sufficient amount of such Red Yeast Rice extract to provide about 9.6 mg of monacolin K, or the daily dose can comprise a sufficient amount of such Red Yeast Rice extract to provide about 12.8 mg monacolin K, or the daily dose can comprise a sufficient amount of such Red Yeast Rice extract to provide about 14.4 mg monacolin K, or the daily dose can comprise a sufficient amount of such Red Yeast Rice extract to provide about 16.0 mg monacolin K. These amounts of monacolin K in the daily dose are to be understood to include any amount or range of amounts between about 6.0 mg and 16.0 mg in increments of 0.01 mg.

The present invention further relates to a method of reducing serum cholesterol, triglycerides or both in a subject comprising administering a daily dosage of a composition of this invention. In some embodiments, the daily dosage comprises those recited above.

In some embodiments, the invention relates to compositions comprising Red Yeast Rice extract comprising greater than 5 wt. % monacolin K, and the use of such compositions to treat a subject. In some embodiments, the Red Yeast Rice extract comprises monacolin K and at least one other monacolin compound. In some embodiments, the monacolin compounds comprise all of the monacolin compounds in Red Yeast Rice.

The compositions of the present invention provide several advantages over the use of lovastatin to reduce cholesterol and triglyceride level in a subject. For instance, the Red Yeast Rice extract is water soluble, whereas lovastatin is not. As noted above, the water insolubility of lovastatin leads to inadequate dissolution in gastrointestinal fluids and, hence, poor absorption, distribution, and targeted organ delivery. While the water solubility of lovastatin can be enhanced, it is believed that the water soluble Red Yeast Rice extract will enter the subjects system easier than lovastatin.

It is also emphasized that Red Yeast Rice extract can produce better lipid reducing results at lower dosages (based on the amount of monacolin in the Red Yeast Rice extract) than lovastatin. This reduces the risk of undesirable and possibly harmful side effects in the subject.

The Red Yeast Rice extract is prepared by fermenting white rice, preferably non-glutinous white rice, with Monascus purpureus strain of yeast by culturing said Monascus purpureus strain in a culture medium comprising rice at a temperature of about 15° C. to about 35° C. for a period of about 2 to about 20 days to provide a crude fermentation product containing Red Yeast Rice; drying said crude fermentation product to obtain Red Yeast Rice, extracting said Red Yeast Rice with a solvent to provide an extract; and drying said extract to remove the solvent and produce Red Yeast Rice extract. The solvent is preferably either aqueous ethanol or water. Other culture media may also be added to the rice. For example, sugar; an additional carbon source chosen from the group consisting of glycerine, malt, and potato juice; and thick beet juice or mixtures thereof may be used. In addition, a defoamer may be added.

In some embodiments, the Red Yeast Rice extract used in the compositions of this invention contains relatively high levels of monacolin K. These high monacolin content Red Yeast Rice extracts can be prepared as described above with the fermentation step being continued to increase the monacolin K level to the desired amount.

In some embodiments, the Red Yeast Rice extract used in the compositions of the present invention contains greater than 5 wt. % monacolin K, for example 5.5 wt. % to about 25 wt. % monacolin K.

### Comparison between Red Yeast Rice Extract and Lovastatin

Red Yeast Rice extract contains monacolin K, the lactone form of the statin drug Mevacor® (lovastatin). Red Yeast Rice extract has been tested in clinical trials at daily dosages of 1.2g and 2.4g. The monacolin K content of the Red Yeast Rice extract used in the clinical trials was 0.20% of the Red Yeast Rice extract. The monacolin K dose was therefore 2.4 to 4.8 mg/day. At 2.4 mg/day of monacolin K, the total cholesterol, LDL cholesterol, and triglycerides dropped by 23%, 31%, and 34% respectively. At 4.8 mg/day, the reduction was 17%, 23%, and 16% respectively (see Monograph by Thorne Research Inc., Alternative Medicine Review, 2004, 9:1).

Lovastatin has been shown to have a cholesterol lowering effect in doses ranging from 5 to 80 mg/day (see Bates et al., “Effectiveness of low dosage lovastatin in lowering serum cholesterol. Experience with 56 patients.” Archives of Internal Medicine 1990, 150:1947-1950). A study was performed to show the effectiveness of low-dose lovastatin in lowering serum cholesterol (see Heber et al., “Cholesterol lowering effects of proprietary Chinese red yeast rice dietary supplement,” Ann J Clin Vutr 1999, 69:231-236). Fifty-six patients were given 20 mg/day of lovastatin for 24 weeks. Total cholesterol fell by 26% and triglycerides fell by 12%.

Mevacor® (lovastatin) in its package insert reported extensive clinical trials at dosages of 10, 20, and 40 mg/day. Total cholesterol was reduced in the range from 16-24%, LDL was reduced by 21-32%, and triglycerides were reduced by 10 to 6% (higher reduction observed at lower dosage).

It should be noted that Red Yeast Rice extract at a dosage of 2.4 mg/day of monacolin K produced better lipid reducing results than Mevacor at 10-40 mg/day. It is, therefore, unlikely that the lipid lowering effects with Red Yeast Rice result from the monacolin K content alone of Red Yeast Rice, but are probably attributable in whole or in part to the other monacolins, sterols (beta-sitosterol, campesterol, sigmasterol, and sapogenin), isoflavones, and monounsaturated fatty acids Red Yeast Rice extract (see Durington et al., “An omega-3 polyunsaturated fatty acid concentration administered for one year decreased triglycerides in simvastin treated patients with CM,” Heart 2001, 85(5) 544-548) This is a particular advantage since the lower dosage of Red Yeast Rice extract containing HMG-CoA reductase inhibitor contributes to reduced side effects as well.

The compositions of this invention are suitable for therapeutic and/or nutritional purposes in treating a subject in need of such treatment. As used herein, the term “subject” includes, but is not limited to, a non-human animal, such as a cow, monkey, horse, sheep, pig, chicken, turkey, quail, cat, dog, mouse, rat, rabbit, or guinea pig; and a human.

The amount of the composition of the invention that is effective will vary depending upon the condition being treated, and can be determined by standard clinical techniques. In addition, in vitro or in vivo assays can optionally be employed to help identify optimal dosage ranges. The precise dose to be employed will also depend on the relative amounts of the components of the compositions of the invention, route of administration, and the seriousness of the condition being treated and should be decided according to the judgment of the practitioner and each subject's circumstances.

The compositions of the present invention comprise high-monacolin K content Red Yeast Rice extracts in amounts effective to help reduce LDL cholesterol and/or triglycerides in a subject. The phrase “present in amounts effective to help reduce LDL cholesterol and/or triglycerides in a subject” as used herein means that the Red Yeast Rice extract is used in an amount effective for a therapeutic, preventive or nutritional activity in a subject that promotes or supports reduction of LDL cholesterol and/or triglycerides in the subject. By “promote or support reduction of LDL cholesterol and/or triglycerides in a subject” is meant the compositions help lower (or at least help maintain) LDL cholesterol and/or triglyceride levels in the subject.

The form in which the composition of the invention is administered to the subject is not critical. Typically, the composition is administered as a liquid, as a dispersion or in a capsule. Typically, the composition is administered in the form of individual doses.

In some embodiments, the composition of the invention is administered in the form of a daily dose. However, depending on the severity of the condition being treated, this may not be required, and the period between administration of the doses may be longer than one day. In addition, the term “administer” includes both the case where a third party administers the dose to the subject and the case where the subject self-administers the dose.

Although the present invention has been described in considerable detail with reference to certain versions thereof, other versions are possible. Therefore the spirit and scope of the appended claims should not be limited to the versions presented herein.

What is claimed is:

1. A composition comprising Red Yeast Rice extract comprising greater than 5 wt. % monacolin K.

2. The composition of claim 1 wherein the Red Yeast Rice extract comprises 5.5 wt. % to about 25 wt. % monacolin K.

3. The composition of claim 1 wherein a daily dose of the composition comprises a sufficient amount of Red Yeast Rice extract to provide at least about 6.0 mg of monacolin K.

4. The composition of claim 1 wherein a daily dose of the composition comprises a sufficient amount of Red Yeast Rice extract to provide about 6.4 mg of monacolin K.

5. The composition of claim 1 wherein a daily dose of the composition comprises a sufficient amount of Red Yeast Rice extract to provide about 9.6 mg of monacolin K.

6. The composition of claim 1 wherein a daily dose of the composition comprises a sufficient amount of Red Yeast Rice extract to provide about 12.8 mg of monacolin K.

7. The composition of claim 1 wherein a daily dose of the composition comprises a sufficient amount of Red Yeast Rice extract to provide about 14.4 mg of monacolin K.

8. The composition of claim 1 wherein a daily dose of the composition comprises a sufficient amount of Red Yeast Rice extract to provide about 16.0 mg of monacolin K.

9. A method of reducing serum cholesterol, triglycerides or both in a subject comprising administering to the subject an effective amount of a dosage comprising Red Yeast Rice extract comprising greater than 5 wt. % monacolin K.

10. The method of claim 9 wherein the Red Yeast Rice extract comprises 5.5 wt. % to about 25 wt. % monacolin K.

11. The method of claim 9 wherein a daily dose of the composition comprises a sufficient amount of Red Yeast Rice extract comprising greater than 5 wt. % monacolin K to provide at least about 6.0 mg of monacolin K.

12. The method of claim 9 wherein a daily dose of the composition comprises a sufficient amount of Red Yeast Rice extract comprising greater than 5 wt. % monacolin K to provide about 6.4 mg of monacolin K.

13. The method of claim 9 wherein a daily dose of the composition comprises a sufficient amount of Red Yeast Rice extract comprising greater than 5 wt. % monacolin K to provide about 9.6 mg of monacolin K.

14. The method of claim 9 wherein a daily dose of the composition comprises a sufficient amount of Red Yeast Rice extract comprising greater than 5 wt. % monacolin K to provide about 12.8 mg of monacolin K.

15. The method of claim 9 wherein a daily dose of the composition comprises a sufficient amount of Red Yeast Rice extract comprising greater than 5 wt. % monacolin K to provide about 14.4 mg of monacolin K

16. The method of claim 9 wherein a daily dose of the composition comprises a sufficient amount of Red Yeast Rice extract comprising greater than 5 wt. % monacolin K to provide about 16.0 mg of monacolin K.
